(19) United States
(12) Patent Application Publication
(10) Pub. No.: US 2026/0284649 A1
(43) Pub. Date: Sep. 24, 2026

(54) SELECTIVE SEMI-HYDROGENATION OF ALLENE

(71) Applicant: FIRMENICH SA, SATIGNY (CH)

(72) Inventors: Lionel SAUDAN, Satigny (CH); Gilles ODDON, Satigny (CH)

(21) Appl. No.: 19/480,262

(22) PCT Filed: May 3, 2024
(86) PCT No.: PCT/EP2024/062221

§ 371 Date: Oct. 30, 2025

(30) Foreign Application Priority Data
May 3, 2023 (EP) 23171272.0

(51) Int. Cl. B01J 31/24; C07C 29/17; C07C 33/02

(52) U.S. Cl. CPC B01J 31/2433; C07C 29/17; C07C 33/02; B01J 2231/645; B01J 2531/845

(57) ABSTRACT

The present invention relates to the field of catalytic hydrogenation and, more particularly, to the semi-hydrogenation of an allene into the corresponding alkene in the presence of a homogeneous catalytic system wherein the homogeneous catalytic system comprises a cobalt salt, a bidentate diphosphine ligand and a weakly or non-coordinating mono anion or a reducing agent.

### TECHNICAL FIELD

The present invention relates to the field of catalytic hydrogenation and, more particularly, to the semi-hydrogenation of an allene into the corresponding alkene in the presence of a homogeneous catalytic system wherein the homogeneous catalytic system comprises a cobalt salt, a bidentate diphosphine ligand and a weakly or non-coordinating mono anion or a reducing agent.

### BACKGROUND

Compounds comprising alkene, in particular ω-1 alkene group represent skeletons highly desirables which could be used as such or as key intermediates useful to prepare more complex compounds in different fields such as, among others, perfumery, cosmetic, pharmaceutic or agrochemistry. Said alkene group may be obtained by the reduction of the corresponding triple bond, method suitable in order to favour the formation of the double bond with a cis stereochemistry. However, the access to the corresponding triple bond may be tedious. An alternative approach is the semi-hydrogenation of an allene. Despite the fact that allene are readily accessible, their semi-hydrogenation, and especially the semi-hydrogenation of terminal allene into ω-1 alkene, has been barely disclosed in the literature and suffer from medium to low stereoselectivity and regioselectivity.

So today, there is a need to develop a novel access to alkene group from the semi-hydrogenation of allene while reaching high regioselectivity and controlling the stereoselectivity.

The present invention allows the semi-hydrogenation, using molecular H₂, of an allene into the corresponding alkene wherein said process is carried out in the presence of a homogeneous catalytic system comprising a cobalt salt, a bidentate diphosphine ligand and a weakly or non-coordinating mono anion or a reducing agent. The invention's conditions allow favouring the formation of the cis while reaching a high regioselectivity. To the best of our knowledge, the invention's process has never been reported in the prior art.

### DESCRIPTION OF THE INVENTION

Surprisingly, it has now been discovered that a homogeneous catalytic system comprising a cobalt salt, a bidentate diphosphine ligand and a weakly or non-coordinating mono anion or a reducing agent allows the hydrogenation of an allene with high regio- and stereoselectivity.

So, a first object of the present invention is a process for the semi-hydrogenation, using molecular H₂, of an allene into the corresponding alkene wherein said process is carried out in the presence of a homogeneous catalytic system wherein the homogeneous catalytic system comprises a cobalt salt, a bidentate diphosphine ligand and a weakly or non-coordinating mono anion or a reducing agent.

As the term “bidentate diphosphine ligand” or similar is understood as a ligand coordinating the cobalt metal with two phosphine atoms.

For the sake of clarity, by the term “alkene”, or the similar, it is meant the normal meaning understood by a person skilled in the art, i.e. that the compound obtained by the semi-hydrogenation comprises a double bond which may have a cis configuration corresponding to the Z isomer, a trans configuration corresponding to the E isomer or a mixture thereof. Indeed, the alkene may be in the form of its E or Z isomer or a mixture thereof. In other words, the alkene may be in the form of its E or Z isomer or of a mixture thereof, e.g. the invention process leads to a composition of matter consisting of one or more alkene, having the same chemical structure but differing by the configuration of the double bond. In particular, the alkene can be in the form of a mixture consisting of isomers E and Z and wherein said isomer Z represents at least 50% of the total mixture, or even at least 75% (i.e a mixture Z/E comprised between 75/25 and 100/0).

According to any embodiments of the invention, the homogeneous catalytic system comprises a cobalt in the oxidation state 0 or +1 or +11.

According to a particular embodiment of the invention, the homogeneous catalytic system is a Co(0) or a Co(I) complex of formula

[Co(PP)(L)_{q}](Y)ₓ (I)

wherein PP is a bidentate diphosphine ligand; x is 0 or 1; L is a C₄-C₂₀ hydrocarbon ligand comprising two or three carbon-carbon double bonds optionally comprising one or more of an oxygen and/or halogen atom and/or silicium atom and q is 1; or L is C₂-C₁₅ alkene ligand and q is 3; and Y is a weakly or non-coordinating mono anion.

The term “optionally” is understood that a certain group to be optionally substituted or optionally comprising can or cannot be substituted with a certain functional group or can or cannot comprise a certain atom. The term “one or more” is understood as being substituted with 1 to 7, preferably 1 to 5, preferably 1 to 3 and more preferably 1 to 2 of a certain functional group.

It is understood that by “ . . . hydrocarbon ligand . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynyl group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the term “ . . . a hydrocarbon group, optionally comprising . . . ” it is meant that said hydrocarbon group optionally comprises one two or three oxygen atoms in a form of alcohol, ketone, aldehyde, ether, ester, carboxylic acid, carbonate groups and/or one or two nitrogen atoms in a form of amine or amide groups and/or one sulfur atom in a form of a thiol group. These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain. For example, a —CH₂—CH₂—CHOH—CH₂— group represents a C₄ hydrocarbon group comprising an alcohol group (substitution of a hydrogen atom), i.e. a C₄ hydrocarbon comprising an oxygen atom; a —CH₂—CH₂—COO—CH₂—CH₂CH₂—CH₂— group represents a C₇ hydrocarbon group comprising one ester group (substitution of carbon atoms/insertion into the hydrocarbon chain), i.e. a C₇ hydrocarbon comprising two oxygen atoms and, similarly, a —CH₂—CH₂—O—CH₂—CH₂—O—CH₂—CH₂— group represents a C₆ hydrocarbon group comprising two ether groups, i.e. a C₆ hydrocarbon comprising two oxygen atoms.

It is understood that the ligand L is a neutral ligand and not a anionic ligand such as an allylic ligand.

The term “C₃-C₁₅ alkene ligand” is understood as a ligand comprising one double bond wherein the ligand coordinated to the cobalt metal by the double bond. Said alkene may be linear, cyclic or branched.

According to any embodiments of the invention, x is 1.

According to any embodiments of the invention, q is 1 or q is 3. When q is 3, then L may be a C₂-C₁₂ alkene ligand, particularly, C₂-C₁₀ alkene ligand, particularly, C₂-C₈ alkene ligand, even more particularly a C₂-C₆ alkene ligand. Examples of suitable alkene ligands include, but are not limited to, ethylene, cyclooctene, cyclohexene, prop-1-ene, but-1-ene, but-2-ene, cycloheptene, cyclopentene, cyclododecene, tetramethylethylene, substituted stilbenes (ArCH═CHAr). Preferably, q is 1 and then L may be a linear or branched C₄-C₁₅ hydrocarbon compound comprising two or three carbon-carbon double bonds or a cyclic C₆-C₂₀ hydrocarbon group comprising two or three carbon-carbon double bonds or an arene optionally substituted by one or more of a C₁₋₆ alkyl group, C₁₋₆ alkoxyl group, hydroxy group or halogen atom. Preferably, L may be a cyclic C₆-C₁₅ hydrocarbon group comprising two or three carbon-carbon double bonds, conjugated or not, or an arene optionally substituted by one or more of a C₁₋₄ alkyl group, C₁₋₄ alkoxyl group, hydroxy group or halogen atom. Preferably, L may be a cyclic C₆-C₁₀ hydrocarbon group comprising two or three carbon-carbon double bonds or an arene optionally substituted by one or more of a C₁₋₃ alkyl group, C₁₋₃ alkoxyl group, hydroxy group or halogen atom. Even more preferably, L may be a cyclic C₆-C₁₀ alkadiene group or a arene optionally substituted by one or more of a C₁₋₃ alkyl group, C₁₋₃ alkoxyl group, hydroxy group or halogen atom. Examples of suitable L include, but are not limited to, 1,5-cyclooctadiene (COD), 1,3-cyclooctadiene, norbornadiene, 2,4-dimethyl-1,3-pentadiene, cyclohepta-1,4-diene, cycloheptatriene, cyclooctatriene, 1,1-dimethyl-2,3-dimethylene cyclohexane, dimethyl-divinyl silane or η⁶ ligand selected from the group consisting of (η⁶-C₆H₆), (η⁶-C₆H₅CH₃), (η⁶-C₆H₅OCH₃), (η⁶-C₆H₄(CH₃)₂), (η⁶-C₆H₅CF₃), (η⁶-C₆H₅F), (η⁶-C₆H₄F₂) and (η⁶-C₆H₄(CF₃)₂).

According to any embodiment of the invention, the weakly or non-coordinating mono anion Y is sterically hindered. In particular, the weakly or non-coordinating mono anion Y may be SbF₆⁻ or of formula BR′₄⁻, wherein R′ is a halogen atom or a phenyl group optionally substituted by one to five halogen atoms or methyl or CF₃ groups or of formula Al(OR″)₄⁻ wherein R″ is a C₁₋₆ alkyl optionally substituted by one or more halogen atoms.

In particular, the weakly or non-coordinating coordinating mono anion Y is selected from the group consisting of ⁻SbF₆, ⁻BF₄, ⁻Al(OC₃HF₆)₄, ⁻Al(OC₄F₉)₄ and ⁻B(3,5-bis(trifluoromethyl)phenyl)₄.

According to any embodiment of the invention, the bidentate diphosphine ligand (PP) is an electron rich ligand. Particularly, the bidentate diphosphine ligand (PP) is a C₅-C₅₀ bidentate ligand wherein the coordinating groups are two phosphorous atoms. Particularly, the two phosphine atoms of the bidentate diphosphine ligand (PP) are separated by at least 2 carbon atoms and by at most 4 carbon atoms. Particularly, the bidentate diphosphine ligand (PP) may be of formula

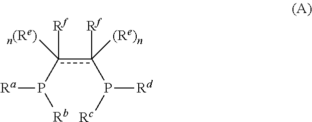

wherein the dotted line is a single bond then n is 1 or the dotted line is a double bond and then n is 0; R^{a}, R^{b}, R^{c} and R^{d}, when taken separately, represent, simultaneously or independently, a linear C₁ to C₈ alkyl, a linear C₂ to C₈ alkenyl, a branched or cyclic C₃ to C₈ alkyl or alkenyl, a C₆ to C₁₀ aryl group, each optionally substituted by one or more of a halogen atom, C₁₋₁₀ alkoxy, hydroxy, halo- or perhalo-hydrocarbon, C₁₋₄ carboxylic ester group; or R^{a} and R^{b}, when taken together, and/or R^{c} and R^{d}, when taken together, form a saturated or unsaturated ring, having 4 to 20 atoms and including the phosphorus atom to which said R^{a} and R^{b} groups or R^{c} and R^{d} are bonded, said ring being optionally substituted by one or more of a halogen atom, C₁₋₁₀ alkoxy, hydroxy, halo- or perhalo-hydrocarbon, C₁₋₄ carboxylic ester, C₆₋₁₀ aryl group; R^{e} and R^{f}, independently from each other, are a hydrogen atom or a C₁₋₈ hydrocarbon group; optionally comprising one or two oxygen or nitrogen atoms; or both R^{f} are taken together and form a C₃₋₁₀ cycloalkyl, a C₅₋₁₀ cycloalkenyl, a C₄₋₁₀ heterocycloalkyl, a C₄₋₁₀ heterocycloalkenyl, a C₄₋₁₀ heteroaryl or a C₆₋₁₀ aryl group, each optionally substituted by one or two halogen atoms, C₁₋₁₀ alkoxy, hydroxy, halo- or perhalo-hydrocarbon, C₁₋₄ carboxylic ester groups; or R^{a} and R^{f} and/or R^{d} and R^{f}, when taken together, form a saturated or unsaturated ring, having 4 to 20 atoms and including the phosphorus atom to which said R^{a} and R^{f} groups and/or R^{d} and R^{f} are bonded, said ring being optionally substituted by one or two halogen atoms, C₁₋₁₀ alkoxy, hydroxy, halo- or perhalo-hydrocarbon, C₁₋₄ carboxylic ester groups.

The terms “alkyl”, “alkenyl” and “alkoxy” are understood as comprising branched and linear alkyl and alkenyl groups except if indicated otherwise. The terms “alkenyl”, “cycloalkenyl” and “heterocycloalkenyl” are understood as comprising 1, 2 or 3 olefinic double bonds, preferably 1 or 2 olefinic double bonds. The terms “cycloalkyl”, “cycloalkenyl”, “heterocycloalkyl”, “heterocycloalkenyl” and “saturated or unsaturated ring” are understood as comprising a monocyclic or fused, spiro and/or bridged bicyclic or tricyclic cycloalkyl, cycloalkenyl, heterocycloalkyl, heterocycloalkenyl groups and saturated or unsaturated ring, preferably monocyclic cycloalkyl, cycloalkenyl, heterocycloalkyl and heterocycloalkenyl groups.

The terms “halo- or perhalo-hydrocarbon” are understood as hydrocarbon group wherein a hydrogen atom or all hydrogen atoms have been replaced by halogen atoms. Example of “halo- or perhalo-hydrocarbon” are CClH₂ or CF₃.

The term “aryl” is understood as comprising any group comprising at least one aromatic group such as phenyl, indenyl, indanyl, tetrahydronaphthalenyl or naphthalenyl group.

The term “heteroaryl is understood as an aromatic group comprising an heteroatom such as pyridyl or furyl group.

For the sake of clarity, by the expression “R^{a} and R^{b}, when taken together, and/or R^{c} and R^{d}, when taken together, form a saturated or unsaturated ring” or “or both R are taken together and form a C₃₋₁₀ cycloalkyl . . . ”, it is meant that the atoms to which both groups are bonded are included into the C₃₋₁₀ cycloalkyl group for instance.

For the sake of clarity, by the expressions “R^{a} and R^{f} and/or R^{d} and R^{f}, when taken together, form a saturated or unsaturated ring, having 4 to 20 atoms . . . ” or “R^{a} and R^{b}, when taken together, and/or R^{c} and R^{d}, when taken together, form a saturated or unsaturated ring, having 4 to 20 atoms . . . ”, it is meant that the 4 to 20 atoms are carbon atoms and optionally one oxygen, sulfur or nitrogen atom.

According to any embodiment of the invention, R^{a}, R^{b}, R^{c} and R^{d}, when taken separately, may be a linear C₁ to C₆ alkyl, a linear C₂ to C₆ alkenyl, a branched or cyclic C₃ to C₆ alkyl or alkenyl, a C₆ to C₁₀ aryl group, each optionally substituted by one or more of a halogen atom, C₁₋₈ alkoxy, hydroxy, halo- or perhalo-hydrocarbon, C₁₋₃ carboxylic ester group. Particularly, R^{a}, R^{b}, R^{c} and R^{d}, when taken separately, may be a linear C₁ to C₆ alkyl, a linear C₂ to C₆ alkenyl, a branched or cyclic C₃ to C₆ alkyl or alkenyl, a C₆ to C₁₀ aryl group, each optionally substituted by one or more of a C₁₋₈ alkoxy, hydroxy or C₁₋₃ carboxylic ester group. Particularly, R^{a}, R^{b}, R^{c} and R^{d}, when taken separately, may be a linear C₁ to C₆ alkyl, a linear C₂ to C₆ alkenyl, a branched or cyclic C₃ to C₆ alkyl or alkenyl, a C₆ to C₁₀ aryl group, each optionally substituted by one or more of a C₁₋₆ alkoxy, hydroxy or C₁₋₃ carboxylic ester group. Particularly, R^{a}, R^{b}, R^{c} and R^{d}, when taken separately, may be a linear C₁ to C₆ alkyl, a linear C₂ to C₆ alkenyl, a branched or cyclic C₃ to C₆ alkyl or alkenyl, a C₆ to C₁₀ aryl group, each optionally substituted by one or more of a C₁₋₄ alkoxy, hydroxy or C₁₋₃ carboxylic ester group. Particularly, R^{a}, R^{b}, R^{c} and R^{d}, when taken separately, may be a linear C₁ to C₆ alkyl, a linear C₂ to C₆ alkenyl, a branched or cyclic C₃ to C₆ alkyl or alkenyl, a C₆ aryl group, each optionally substituted by one or more of a C₁₋₄ alkoxy, hydroxy or C₁₋₃ carboxylic ester group. Particularly, R^{a}, R^{b}, R^{c} and R^{d}, when taken separately, may be a linear C₁ to C₆ alkyl, a linear C₂ to C₆ alkenyl, a branched or cyclic C₃ to C₆ alkyl or alkenyl, a C₆ aryl group, each optionally substituted by one or more of a C₁₋₃ alkoxy, hydroxy or C₁₋₃ carboxylic ester group. Even more particularly, R^{a}, R^{b}, R^{c} and R^{d}, when taken separately, may be a linear C₁ to C₄ alkyl, a linear C₂ to C₄ alkenyl, a branched or cyclic C₅ to C₆ alkyl or alkenyl, a C₆ aryl group, each optionally substituted by one or more of a C₁₋₃ alkoxy, hydroxy or C₁₋₃ carboxylic ester group.

According to any embodiment of the invention, R^{a} and R^{b}, when taken together, and/or R^{c} and R^{d}, when taken together, may be a C₄₋₁₅ alkanediyl group optionally substituted by one or more of a halogen atom, C₁₋₈ alkoxy, hydroxy, halo- or perhalo-hydrocarbon, C₁₋₄ carboxylic ester, C₆₋₁₀ aryl group. Particularly, R^{a} and R^{b}, when taken together, and/or R^{c} and R^{d}, when taken together, may be a C₄₋₁₅ alkanediyl group optionally substituted by one or more of a C₁₋₈ alkoxy, hydroxy, C₁₋₄ carboxylic ester or C₆₋₁₀ aryl group. Particularly, R^{a} and R^{b}, when taken together, and/or R^{c} and R^{d}, when taken together, may be a C₄₋₁₅ alkanediyl group optionally substituted by one or more of a C₁₋₆ alkoxy, hydroxy, C₁₋₃ carboxylic ester or C₆ aryl group. Particularly, R^{a} and R^{b}, when taken together, and/or R^{c} and R^{d}, when taken together, may be a C₄₁₂ alkanediyl group optionally substituted by one or more of a C₁₋₄ alkoxy, hydroxy, C₁₋₃ carboxylic ester or C₆ aryl group. Even more particularly, R^{a} and R^{b}, when taken together, and/or R^{c} and R^{d}, when taken together, may be a C₄₁₂ alkanediyl group.

According to any embodiment of the invention, R^{a} and R^{f} and/or R^{d} and R^{f}, when taken together, form a saturated or unsaturated ring, having 4 to 15 atoms and including the phosphorus atom to which said R^{a} and R^{f} groups and/or R^{d} and R^{f} are bonded, said ring being optionally substituted by one or two halogen atoms, C₁₋₁₀ alkoxy, hydroxy, halo- or perhalo-hydrocarbon, C₁₋₄ carboxylic ester groups. Particularly, R^{a} and R^{f} and/or R^{d} and R^{f}, when taken together, form a saturated or unsaturated ring, having 4 to 10 atoms and including the phosphorus atom to which said R^{a} and R^{f} groups and/or R^{d} and R are bonded, said ring being optionally substituted by one or two halogen atoms, C₁₋₁₀ alkoxy, hydroxy, halo- or perhalo-hydrocarbon, C₁₋₄ carboxylic ester groups. Particularly, R^{a} and R^{b} and/or R^{d} and R^{f}, when taken together, form a saturated or unsaturated ring, having 4 to 10 atoms and including the phosphorus atom to which said R^{a} and R^{f} groups and/or R^{d} and R^{f} are bonded, said ring being optionally substituted by one or two halogen atoms, C₁₋₈ alkoxy, hydroxy, halo- or perhalo-hydrocarbon, C₁₋₃ carboxylic ester groups. Particularly, R^{a} and R^{f} and/or R^{d} and R^{f}, when taken together, form a saturated or unsaturated ring, having 4 to 10 atoms and including the phosphorus atom to which said R^{a} and R^{f} groups and/or R^{d} and R^{f} are bonded, said ring being optionally substituted by one or two halogen atoms, C₁₋₆ alkoxy, hydroxy, halo- or perhalo-hydrocarbon, C₁₋₃ carboxylic ester groups. Even more particularly, R^{a} and R^{f} and/or R^{d} and R^{f}, when taken together, form a saturated or unsaturated ring, having 4 to 10 atoms and including the phosphorus atom to which said R^{a} and R^{f} groups and/or R^{d} and R^{f} are bonded, said ring being optionally substituted by one or two halogen atoms, C₁₋₃ alkoxy, hydroxy, halo- or perhalo-hydrocarbon, C₁₋₃ carboxylic ester groups.

According to any embodiment of the invention, the bidentate diphosphine ligand (PP) is of formula

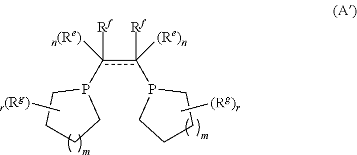

wherein the dotted line, n, R^{e} and R^{f} has the same meaning as defined above; m is 0, 1 or 2; r is 1, 2, 3, 4, 5 or 6 and each R^{g}, independently from each other, represents at least one substituent of the ring and are independently from each other, are a C₁₋₈ alkyl or a C₆₋₁₀ aryl group.

According to any embodiment of the invention, r may be 1, 2 or 3. Particularly, r may be 2 and R⁹ groups may be, relative to the phosphorous atom, two ortho substituents of the ring.

According to any embodiment of the invention, the bidentate diphosphine ligand (PP) is of formula

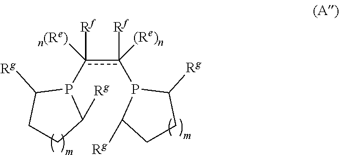

wherein the dotted line, n, R^{e} and R^{f} has the same meaning as defined above; m is 0, 1 or 2 and each R^{g}, independently from each other, are a C₁₋₈ alkyl or a C₆₋₁₀ aryl group.

According to any embodiment of the invention, R^{e} and R^{f}, independently from each other, are a hydrogen atom or a C₁₋₆ hydrocarbon group; optionally comprising one or two oxygen or nitrogen atoms. Particularly, R^{e} and R^{f}, independently from each other, are a hydrogen atom or a C₁₋₆ alkyl group. Particularly, R^{e} and R^{f}, independently from each other, are a hydrogen atom or a C₁₋₄ alkyl group. Particularly, R^{e} and R^{f}, independently from each other, are a hydrogen atom or a C₁₋₃ alkyl group. Particularly, R^{e} and R^{f}, independently from each other, are a hydrogen atom or a methyl or ethyl group. Particularly, R^{e} and R^{f}, independently from each other, are a hydrogen atom or a methyl group. Even more particularly, R^{e} and R^{f}, independently from each other, are a hydrogen atom.

According to any embodiment of the invention, both R^{f} are taken together and form a C₃₋₈ cycloalkyl, a C₅₋₈ cycloalkenyl, a C₄₋₈ heterocycloalkyl, a C₄₋₈ heterocycloalkenyl, a C₄₋₈ heteroaryl or a C₆ aryl group, each optionally substituted by one or two halogen atoms, C₁₋₁₀ alkoxy, hydroxy, halo- or perhalo-hydrocarbon, C₁₋₄ carboxylic ester groups. Particularly, According to any embodiment of the invention, both R^{f} are taken together and form a C₃₋₈ cycloalkyl, a C₅₋₈ cycloalkenyl, a C₄₋₈ heterocycloalkyl, a C₄₋₈ heterocycloalkenyl, a C₄₋₈ heteroaryl or a C₆ aryl group, each optionally substituted by one or two halogen atoms, C₁₋₈ alkoxy, hydroxy, halo- or perhalo-hydrocarbon, C₁₋₃ carboxylic ester groups. Particularly, According to any embodiment of the invention, both R^{f} are taken together and form a C₃₋₈ cycloalkyl, a C₅₋₈ cycloalkenyl, a C₄₋₈ heterocycloalkyl, a C₄₋₈ heterocycloalkenyl, a C₄₋₈ heteroaryl or a C₆ aryl group, each optionally substituted by one or two halogen atoms, C₁₋₆ alkoxy, hydroxy, halo- or perhalo-hydrocarbon, C₁₋₃ carboxylic ester groups. Particularly, both R^{f} are taken together and form a C₃₋₈ cycloalkyl, a C₅₋₈ cycloalkenyl, a C₄₋₈ heterocycloalkyl, a C₄₋₈ heterocycloalkenyl, a C₄₋₈ heteroaryl or a C₆ aryl group, each optionally substituted by one or two halogen atoms, C₁₋₄ alkoxy, hydroxy, halo- or perhalo-hydrocarbon, C₁₋₃ carboxylic ester groups. Particularly, both R^{f} are taken together and form a C₃₋₈ cycloalkyl, a C₅₋₈ cycloalkenyl, a C₄₋₈ heterocycloalkyl, a C₄₋₈ heterocycloalkenyl, a C₄₋₈ heteroaryl or a C₆ aryl group, each optionally substituted by one or two halogen atoms, C₁₋₃ alkoxy, hydroxy, halo- or perhalo-hydrocarbon, C₁₋₃ carboxylic ester groups. Particularly, both R^{f} are taken together and form a C₄₋₈ cycloalkyl, a C₅₋₈ cycloalkenyl, a C₅₋₇ heterocycloalkyl, a C₅₋₇ heterocycloalkenyl, a C₅₋₇ heteroaryl or a C₆ aryl group, each optionally substituted by one or two halogen atoms, C₁₋₃ alkoxy, hydroxy, halo- or perhalo-hydrocarbon, C₁₋₃ carboxylic ester groups. Particularly, both R^{f} are taken together and form a C₅₋₇ cycloalkyl, a C₅₋₇ cycloalkenyl, or a C₆ aryl group, each optionally substituted by one or two halogen atoms, C₁₋₃ alkoxy, hydroxy, halo- or perhalo-hydrocarbon, C₁₋₃ carboxylic ester groups. Particularly, both R^{f} are taken together and form a C₆ cycloalkyl, a C₆ cycloalkenyl, or a C₆ aryl group, each optionally substituted by one or two halogen atoms, C₁₋₃ alkoxy, hydroxy, halo- or perhalo-hydrocarbon, C₁₋₃ carboxylic ester groups. Even more particularly, both R^{f} are taken together and form a C₆ aryl group.

According to any embodiment of the invention, m is 1.

According to any embodiment of the invention, R⁹, independently from each other, are a C₃₋₈ branched alkyl or a phenyl group. Particularly, R⁹, independently from each other, are a C₃₋₆ branched alkyl or a phenyl group. Even more particularly, R⁹, independently from each other, are an isopropyl or phenyl group.

Examples of suitable bidentate diphosphine ligands (PP) include, but are not limited to 1,2-bis(2,5-diisopropylphospholan-1-yl)benzene, 1,2-bis(2,5-diisopropylphospholan-1-yl)ethane, 1,2-bis(2,5-diphenylphospholan-1-yl)ethane, 1,2-bis(2,5-diphenylphospholan-1-yl)benzene, 1,2-bis(dimethylphosphaneyl)ethane, 1,2-bis(diethylphosphaneyl)ethane, 1,2-bis(diisopropylphosphaneyl)ethane, 1,2-bis(dicyclopentylphosphaneyl)ethane, 1,2-bis(dicyclohexylphosphaneyl)ethane.

Examples of suitable Co(I) complex of formula (I) include, but are not limited to, [Co(1,2-bis(2,5-diisopropylphospholan-1-yl)benzene)(η⁶-C₆H₆)]Al(OC₄F₉)₄, [Co(1,2-bis(2,5-diisopropylphospholan-1-yl)ethane)(η⁶-C₆H₆)]Al(OC₄F₉)₄, [Co(1,2-bis(2,5-diphenylphospholan-1-yl)ethane)(η⁶-C₆H₆)]Al(OC₄F₉)₄, Co(1,2-bis(2,5-diisopropylphospholan-1-yl)benzene(η⁶-C₆H₅CH₃)]Al(OC₄F₉)₄, [Co(1,2-bis(2,5-diisopropylphospholan-1-yl)ethane(η⁶-C₆H₅CH₃)]Al(OC₄F₉)₄, [Co(1,2-bis(2,5-diphenylphospholan-1-yl)ethane)(η⁶-C₆H₅CH₃)]Al(OC₄F₉)₄, Co(1,2-bis(2,5-diisopropylphospholan-1-yl)benzene(η⁶-C₆H₅F)]Al(OC₄F₉)₄, [Co(1,2-bis(2,5-diisopropylphospholan-1-yl)ethane(η⁶-C₆H₅F)]Al(OC₄F₉)₄, [Co(1,2-bis(2,5-diphenylphospholan-1-yl)ethane)(η⁶-C₆H₅F)]Al(OC₄F₉)₄, [Co(1,2-bis(2,5-diisopropylphospholan-1-yl)benzene)(η⁶-C₆H₆)]B(3,5-bis(trifluoromethyl)phenyl)₄, [Co(1,2-bis(2,5-diisopropylphospholan-1-yl)ethane)(η⁶-C₆H₆)]B(3,5-bis(trifluoromethyl)phenyl)₄, [Co(1,2-bis(2,5-diphenylphospholan-1-yl)ethane)(η⁶-C₆H₆)]B(3,5-bis(trifluoromethyl)phenyl)₄, Co(1,2-bis(2,5-diisopropylphospholan-1-yl)benzene(η⁶-C₆H₅CH₃)]B(3,5-bis(trifluoromethyl)phenyl)₄, [Co(1,2-bis(2,5-diisopropylphospholan-1-yl)ethane(η⁶-C₆H₅CH₃)]B(3,5-bis(trifluoromethyl)phenyl)₄, [Co(1,2-bis(2,5-diphenylphospholan-1-yl)ethane) (η⁶-C₆H₅CH₃)]B(3,5-bis(trifluoromethyl)phenyl)₄, Co(1,2-bis(2,5-diisopropylphospholan-1-yl)benzene(η⁶-C₆H₅F)]B(3,5-bis(trifluoromethyl)phenyl)₄, [Co(1,2-bis(2,5-diisopropylphospholan-1-yl)ethane(η⁶-C₆H₅F)]B(3,5-bis(trifluoromethyl)phenyl)₄ or [Co(1,2-bis(2,5-diphenylphospholan-1-yl)ethane) (η⁶-C₆H₅F)]B(3,5-bis(trifluoromethyl)phenyl)₄.

According to any embodiment of the invention, Co(I) complex of formula (I) can be prepared and isolated prior to their use in the process according to the general methods described in the literature such as in ACS Catalysis 2022, 12, 4680-4687.

Moreover, the complexes can be prepared in situ, by several methods, in the hydrogenation medium, without isolation or purification, just before their use.

One of the possible procedures to advantageously prepare in situ a complex of the invention consists in reacting an appropriate cobalt complex of formula [Co(L)_{z}]Y, wherein L and Y have the same meaning as defined above and z is 1 or 2, with a bidentate diphosphine ligands (PP) as defined above, to give a solution of a catalyst according to formula (I).

According to any embodiment of the invention, Co(I) complex of formula (I) can be added to the reaction medium in a large range of concentrations. As non-limiting examples, one can cite as complex concentration values those ranging from 1 ppm to 50000 ppm, relative to the amount of substrate. Preferably, the complex concentration will be comprised between 10 and 10000 ppm. Preferably, the complex concentration will be comprised between 10 and 5000 ppm. Even more preferably, the complex concentration will be comprised between 50 and 5000 ppm. It goes without saying that the optimum concentration of complex will depend, as the person skilled in the art knows, on the nature of the latter, on the nature of the substrate and on the pressure of H₂ used during the process, as well as the desired time of reaction.

According to a particular embodiment of the invention, the homogeneous catalytic system comprises
a) at least a salt of formula MY wherein M is a alkali metal or an alkaline earth metal and Y is a weakly or non-coordinating mono anion; and
b) a Co(I) complex of formula

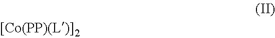

wherein PP is a bidentate diphosphine ligand and L′ is a halogen atom, a C₁₋₁₈ alkoxide, a C₁₋₈ carboxylate or β-diketonate group.

The term “β-diketonate” is understood as a ligand comprising a C(═O)—CH═C(O⁻) group. Particularly, the β-diketonate is of formula R²—C(═O)—CH═C(O⁻)—R³ wherein R² and R³, independently from each other, are a C₁₋₆ alkyl group, particularly a C₁₋₄ alkyl group, even more particularly a methyl, propyl, isopropyl or a terbutyl group. Non-limiting example of β-diketonate may include 4-oxopent-2-en-2-olate, 2,2-dimethyl-5-oxohex-3-en-3-olate, 2,6-dimethyl-5-oxohept-3-en-3-olate or 2,2,6,6-tetramethyl-5-oxohept-3-en-3-olate.

According to any embodiment of the invention, the alkali metal M may be selected from the group consisting of lithium, sodium and potassium and the alkaline earth metal may be magnesium. The non-coordinating mono anion Y has the same meaning as defined above. Particularly, MY may be selected from the group consisting of NaBF₄, LiBF₄, KBF₄, NaSbF₆, LiSbF₆, KSbF₆, NaB(3,5-bis(trifluoromethyl)phenyl)₄, LiB(3,5-bis(trifluoromethyl)phenyl)₄, KB(3,5-bis(trifluoromethyl)phenyl)₄, NaB((pentafluoro)phenyl)₄, LiAl(OC₄F₉)₄, NaAl(OC₄F₉)₄ and a mixture thereof. Preferably, MY may be selected from the group consisting of NaB(3,5-bis(trifluoromethyl)phenyl)₄, LiB(3,5-bis(trifluoromethyl)phenyl)₄ and KB(3,5-bis(trifluoromethyl)phenyl)₄.

The salt of formula MY can be added into the reaction medium of the invention's process in a large range of concentrations. As non-limiting examples, one can cite, as salt of formula MY concentration values those ranging from 1 to 100 equivalents, relative to the total amount of the Co(I) complex of formula (II). Particularly, the salt of formula MY concentration may be comprised between 1 to 50 equivalents, relative to the total amount of the Co(I) complex of formula (II). Even more particularly, the salt of formula MY concentration may be comprised between 1 to 50 equivalents, relative to the total amount of the Co(I) complex of formula (II). It goes without saying that the process works also with more salt of formula MY. However the optimum concentration of salt of formula MY will depend, as the person skilled in the art knows, on the nature of the latter, on the nature of the allene, on the temperature and on the desired time of reaction.

According to any embodiment of the invention, L′ may be a halogen atom, a C₁₋₁₅ alkoxide, a C₁₋₆ carboxylate or a β-diketonate group. Particularly, L′ may be a halogen atom, a C₁₋₁₂ alkoxide, a C₁₋₆ carboxylate or a β-diketonate group. Particularly, L′ may be a halogen atom, a C₁₋₁₀ alkoxide, a C₁₋₆ carboxylate or a β-diketonate group. Particularly, L′ may be a halogen atom, a C₁₋₈ alkoxide, a C₁₋₆ carboxylate or a β-diketonate group. Particularly, L′ may be a halogen atom, a C₁₋₆ alkoxide, a C₁₋₆ carboxylate or a β-diketonate group. Particularly, L′ may be a halogen atom, a C₁₋₄ alkoxide, a C₁₋₄ carboxylate or a β-diketonate group. Particularly, L′ may be a halogen atom, a C₁₋₃ alkoxide, a C₁₋₄ carboxylate or a β-diketonate group. Particularly, L′ may be a chloride or bromide atom, an acetate, a pivalate or a 4-oxopent-2-en-2-olate group. Even more particularly, L′ may be chloride atom.

According to any embodiment of the invention, the bidentate diphosphine ligand (PP) has the same meaning as defined above.

Examples of suitable Co(I) complex of formula (II) include, but are not limited to, [Co(1,2-bis(2,5-diisopropylphospholan-1-yl)benzene)(Cl)]₂, [Co(1,2-bis(2,5-diisopropylphospholan-1-yl)ethane)(Cl)]₂ or [Co(1,2-bis(2,5-diphenylphospholan-1-yl)ethane)(Cl)]₂.

According to any embodiment of the invention, Co(I) complex of formula (II) can be prepared and isolated prior to their use in the process according to the general methods described in the literature such as in Science 2018, 360, 888-893.

Moreover, the complexes can be prepared in situ, by several methods, in the hydrogenation medium, without isolation or purification, just before their use as reported in Science 2018, 360, 888-893.

According to any embodiment of the invention, Co(I) complex of formula (II) can be added to the reaction medium in a large range of concentrations. As non-limiting examples, one can cite as complex concentration values those ranging from 1 ppm to 50000 ppm, relative to the amount of substrate. Preferably, the complex concentration will be comprised between 10 and 10000 ppm. Preferably, the complex concentration will be comprised between 10 and 5000 ppm. Even more preferably, the complex concentration will be comprised between 50 and 5000 ppm. It goes without saying that the optimum concentration of complex will depend, as the person skilled in the art knows, on the nature of the latter, on the nature of the substrate and on the pressure of H₂ used during the process, as well as the desired time of reaction.

According to any embodiment of the invention, the homogeneous catalytic system comprises
a) a reducing agent selected from the group consisting of Zn metal, LiBHEt₃, NaBH₄ and NaBHEt₃; and
b) a Co(II) complex of formula

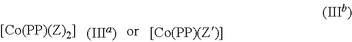

wherein PP is a bidentate diphosphine ligand; Z is a halogen atom, a C₁₋₄ alkyl group optionally substituted by a trimethylsilyl group, a β-diketonate group, a sulfonate group, a phosphate group, a C₁₋₁₈ alkoxide group, a C₁₋₈ carboxylate group or a phenolate group optionally substituted by one or three C₁₋₈ alkyl groups, C₃₋₈ cycloalkyl groups, halogen atoms, C₁₋₃ alkoxy groups, halo- or perhalo-hydrocarbon groups or C₁₋₃ carboxylic ester groups, and Z′ is a carbonate or a sulphate group.

According to any embodiment of the invention, the bidentate diphosphine ligand (PP) has the same meaning as defined above.

According to any embodiment of the invention, the Co(II) complex is a complex of formula (III^{a}) as defined above.

According to any embodiment of the invention, Z may be a halogen atom, a C₁₋₃ alkyl group optionally substituted by a trimethylsilyl group, a C₁₋₁₅ alkoxide, a C₁₋₆ carboxylate or a β-diketonate group. Particularly, Z may be a halogen atom, a C₁₋₂ alkyl group optionally substituted by a trimethylsilyl group, a C₁₋₁₂ alkoxide, a C₁₋₆ carboxylate or a β-diketonate group. Particularly, Z may be a halogen atom, a methyl group optionally substituted by a trimethylsilyl group, a C₁₋₁₀ alkoxide, a C₁₋₆ carboxylate or a β-diketonate group. Particularly, Z may be a halogen atom, a CH₂SiMe₃ group, a C₁₋₈ alkoxide, a C₁₋₆ carboxylate or a β-diketonate group. Particularly, Z may be a halogen atom, a C₁₋₆ alkoxide, a C₁₋₆ carboxylate or a β-diketonate group. Particularly, Z may be a halogen atom, a C₁₋₄ alkoxide, a C₁₋₄ carboxylate or a β-diketonate group. Particularly, Z may be a halogen atom, a C₁₋₃ alkoxide, a C₁₋₄ carboxylate or a β-diketonate group. Particularly, Z may be a chloride or bromide atom, an acetate, a pivalate or a 4-oxopent-2-en-2-olate group. Even more particularly, Z may be chloride atom.

The reducing agent can be added into the reaction medium of the invention's process in a large range of concentrations. As non-limiting examples, one can cite, as reducing agent concentration values those ranging from 0.01 mol % to 10 mol %, relative to the total amount of the allene. Particularly, the reducing agent concentration may be comprised between 0.5 mol % to 5 mol %. It goes without saying that the process works also with more reducing agent. However the optimum concentration of reducing agent Y will depend, as the person skilled in the art knows, on the nature of the latter, on the nature of the allene, on the temperature and on the desired time of reaction.

Examples of suitable Co(II) complex of formula (III^{a}) include, but are not limited to, [Co(1,2-bis(2,5-diisopropylphospholan-1-yl)benzene)(Cl)₂], [Co(1,2-bis(2,5-diisopropylphospholan-1-yl)ethane)(Cl)₂] or [Co(1,2-bis(2,5-diphenylphospholan-1-yl)ethane)(Cl)₂].

According to any embodiment of the invention, Co(II) complex of formula (III^{a}) or (III^{b}) can be prepared and isolated prior to their use in the process according to the general methods described in the literature such as in A. R. Hermes, G. S. Girolami Inorg. Chem. 1988, 27, 1775-1781 or in M. R. Friedfeld, G. W. Margulieux, B. A. Schaefer, P. J. Chirik J. Am Chem. Soc. 2014, 136, 13178-13181.

Moreover, the complexes can be prepared in situ, by several methods, in the hydrogenation medium, without isolation or purification, just before their use, such as by using the cobalt salt: CoCl₂ and the diphosphine.

According to any embodiment of the invention, Co(II) complex of formula (III^{a}) or (III^{b}) can be added to the reaction medium in a large range of concentrations. As non-limiting examples, one can cite as complex concentration values those ranging from 1 ppm to 50000 ppm, relative to the amount of substrate. Preferably, the complex concentration will be comprised between 10 and 10000 ppm. Even more preferably, the complex concentration will be comprised between 10 and 5000 ppm. It goes without saying that the optimum concentration of complex will depend, as the person skilled in the art knows, on the nature of the latter, on the nature of the substrate and on the pressure of H₂ used during the process, as well as the desired time of reaction.

The complex of formula (I) represents, in general for practical reasons, a preferred embodiment of the invention.

According to any embodiment of the invention, the invention's process is the 1,2-hydrogenation of a terminal allene into the corresponding ω-1 alkene.

According to any embodiment of the invention, the terminal allene is of formula

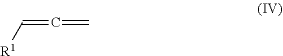

Wherein R¹ is a C₁₋₃₀ hydrocarbon optionally comprising one to three oxygen atoms and/or one to two nitrogen atoms and/or one sulphur atom.

According to any embodiment of the invention, the ω-1 alkene is of formula

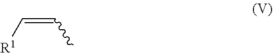

Wherein R¹ have the same meaning as defined above.

For the sake of clarity, by the wavy bond in compound of formula (V), or the similar, it is meant the normal meaning understood by a person skilled in the art, i.e. that the double bond may have a cis configuration corresponding to the Z isomer, a trans configuration corresponding to the E isomer or a mixture thereof.

According to any one of the above embodiments of the invention, the allene is a C₄-C₃₀ compound of formula (IV), particularly, C₅-C₂₀ compound of formula (IV), even more particularly, C₅-C₁₅ compound of formula (IV).

According to any embodiments of the invention, R¹ may be a linear, branched or cyclic C₁-C₃₀ alkyl or alkenyl group, optionally substituted. Particularly, R¹ may be a linear, branched or cyclic C₄-C₃₀ alkyl or alkenyl group, optionally substituted. Particularly, R¹ may be a linear, branched or cyclic C₄-C₂₀ alkyl or alkenyl group, optionally substituted.

Particularly, R¹ may be a linear, branched or cyclic C₅-C₂₀ alkyl or alkenyl group, optionally substituted. Even more particularly, R¹ may be a linear, branched or cyclic C₅-C₁₅ alkyl or alkenyl group, optionally substituted.

Furthermore, according to a yet further embodiment, when R¹ represents an alkenyl group then the carbon-carbon double bond is not terminal and is not conjugated with allene.

Possible substituents of R¹ are one, two or three OR^{h} or R^{h} group, one S(═O)R^{h} or one SO₂R^{h} or one or two NR^{h}₂ wherein R^{h} is a hydrogen atom, a C₁ to C₁₀ cyclic, linear or branched alkyl, or alkenyl group, preferably a C₁ to C₄ linear or branched alkyl or alkenyl group. As other possible substituents one may also cite a group COOR^{h}.

Non-limiting examples of compound of formula (IV) are 4,8-dimethylnona-1,2,7-trien-4-ol, 1-(p-tolyl)buta-2,3-dien-1-ol, 1-cyclohexylbuta-2,3-dien-1-ol, undeca-1,2-dien-4-ol, (E)-5-methylocta-1,2,5-trien-4-ol, 1-phenylhexa-4,5-dien-3-ol, (6R)-6,10-dimethylundeca-1,2,9-trien-4-ol, 2-phenylpenta-3,4-dien-2-ol, 3-phenylhexa-4,5-dien-3-ol, 2-methyl-3-phenylhexa-4,5-dien-3-ol, 4-methyldeca-1,2-dien-4-ol, 3-methyl-1-phenylhexa-4,5-dien-3-ol, 4,4-dimethyl-1-(propa-1,2-dien-1-yl)cyclohexan-1-ol, 1-(propa-1,2-dien-1-yl)cyclopentan-1-ol, 1-(propa-1,2-dien-1-yl)cyclohexan-1-ol, 1-(propa-1,2-dien-1-yl)cyclooctan-1-ol, 2-(propa-1,2-dien-1-yl)decahydronaphthalen-2-ol or (4aS,8aS)-2-(propa-1,2-dien-1-yl)decahydronaphthalen-2-ol.

According to any embodiment of the invention, the terminal allene of formula (IV) can be prepared according to the general methods described in the literature.

Non-limiting examples of compound of formula (V) are (Z)-4,8-dimethylnona-2,7-dien-4-ol, (Z)-2-phenylpent-3-en-2-ol, (Z)-3-phenylhex-4-en-3-ol, (Z)-2-methyl-3-phenylhex-4-en-3-ol, 4-methyldeca-1,2-dien-4-ol, (Z)-3-methyl-1-phenylhex-4-en-3-ol, (Z)-4,4-dimethyl-1-(prop-1-en-1-yl)cyclohexan-1-ol, (Z)-1-(prop-1-en-1-yl)cyclopentan-1-ol, (Z)-1-(prop-1-en-1-yl)cycloheptan-1-ol, (Z)-2-(prop-1-en-1-yl)decahydronaphthalen-2-ol, (4aS,8aS)-2-((Z)-prop-1-en-1-yl)decahydronaphthalen-2-ol, (Z)-1-(prop-1-en-1-yl)cyclohexan-1-ol, (Z)-1-(prop-1-en-1-yl)cyclooctan-1-ol, (Z)-1-(p-tolyl)but-2-en-1-ol, (Z)-1-cyclohexylbut-2-en-1-ol, (2Z,5E)-5-methylocta-2,5-dien-4-ol, (Z)-1-phenylhex-4-en-3-ol, (6R,Z)-6,10-dimethylundeca-2,9-dien-4-ol or (Z)-undec-2-en-4-ol.

According to any embodiment of the invention, the process of the invention is perform in absence of base or additive.

The hydrogenation reaction can be carried out in the presence or absence of a solvent. When a solvent is required or used for practical reasons, then any solvent current in hydrogenation reactions can be used for the purposes of the invention. Non-limiting examples include aromatic solvents such as toluene, fluoro-benzene, trifluoro-toluene, ortho-difluoro-benzene, ortho-dichloro-benzene, chlorobenzene or xylene, hydrocarbon solvents such as hexane or cyclohexane, ethers such as tetrahydrofuran, methyltetrahydrofuran or MTBE, polar solvents such as primary or secondary alcohols such as isopropanol or ethanol, or mixtures thereof. The choice of the solvent is a function of the nature of the complex and the person skilled in the art is well able to select the solvent most convenient in each case to optimize the hydrogenation reaction.

In the hydrogenation process of the invention, the reaction can be carried out at a H₂ pressure comprised between 2⁵ Pa and 100×10⁵ Pa (1 to 100 bars) or even more if desired. Again, a person skilled in the art is well able to adjust the pressure as a function of the catalyst load and of the dilution of the substrate in the solvent. As examples, one can cite typical pressures of 2 to 10×10⁵ Pa (2 to 10 bars).

The temperature at which the hydrogenation can be carried out is comprised between 0° C. and 80° C., more preferably in the range of between 20° C. and 50° C. Of course, a person skilled in the art is also able to select the preferred temperature as a function of the melting and boiling point of the starting and final products as well as the desired time of reaction or conversion.

The invention's process may carried out under batch or continuous conditions.

Another aspect of the invention is the use, in the semi-hydrogenation process as defined above, of compound being selected from the group consisting of 4,8-dimethylnona-1,2,7-trien-4-ol, 1-(p-tolyl)buta-2,3-dien-1-ol, 1-cyclohexylbuta-2,3-dien-1-ol, undeca-1,2-dien-4-ol, (E)-5-methylocta-1,2,5-trien-4-ol, 1-phenylhexa-4,5-dien-3-ol, (6R)-6,10-dimethylundeca-1,2,9-trien-4-ol, 2-phenylpenta-3,4-dien-2-ol, 3-phenylhexa-4,5-dien-3-ol, 2-methyl-3-phenylhexa-4,5-dien-3-ol, 4-methyldeca-1,2-dien-4-ol, 3-methyl-1-phenylhexa-4,5-dien-3-ol, 4,4-dimethyl-1-(propa-1,2-dien-1-yl)cyclohexan-1-ol and 1-(propa-1,2-dien-1-yl)cyclopentan-1-ol, 1-(propa-1,2-dien-1-yl)cyclohexan-1-ol, 1-(propa-1,2-dien-1-yl)cyclooctan-1-ol and 2-(propa-1,2-dien-1-yl)decahydronaphthalen-2-ol.

Some compound of formula (IV) is a novel compound and present a number of advantages as explained above and shown in the examples. Therefore, another object of the present invention is a compound for use in a semi-hydrogenation invention's process, wherein the compounds is selected from the group consisting of 4,8-dimethylnona-1,2,7-trien-4-ol, (E)-5-methylocta-1,2,5-trien-4-ol, (6R)-6,10-dimethylundeca-1,2,9-trien-4-ol, 2-methyl-3-phenylhexa-4,5-dien-3-ol, 1-(propa-1,2-dien-1-yl)cyclohexan-1-ol, 1-(propa-1,2-dien-1-yl)cyclooctan-1-ol, 2-(propa-1,2-dien-1-yl)decahydronaphthalen-2-ol and (4aS,8aS)-2-(propa-1,2-dien-1-yl)decahydronaphthalen-2-ol.

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). The preparation of precatalysts and ligands solutions were carried out under an inert atmosphere (Argon) using standard Schlenk techniques. The solvents were dried by conventional procedures and distilled under an argon atmosphere. NMR spectra were recorded at 20° C. on Bruker AV 300, AV 400, or AV 500 MHz spectrometers. Chemical shifts are reported in ppm relative to solvent signals (chloroform, δ_{H}=7.26 ppm, δ_{C}=77.0 ppm). The signal assignment was ensured by recording ¹H,¹H-COSY, -NOESY, ¹³C,¹H-HSQC and -HMBC experiments. Gas chromatography was performed on an Agilent 6850 Series equipped with a DB-1 or DB-Wax columns (10 m×0.1 mm ID, 0.1 μm film). The cobalt complexes were prepared according to literature procedures, see a) S. C. Meier, A. Holz, J. Kulenkampff, A. Schmidt, D. Kratzert, D. Himmel, D. Schmitz, E.-W. Scheidt, W. Scherer, C. Bülow, M. Timm, R. Lindblad, S. T. Akin, V. Zamudio-Bayer, B. von Issendorff, M. A. Duncan, J. T. Lau, and I. Krossing Angew. Chem. Int. Ed. 2018, 57, 9310-9314. b) C. S. MacNeil, H. Zhong, T. P. Pabst, M. Shevlin, P. J. Chirik ACS Catalysis 2022, 12, 4680-4687. c) M. R. Friedfeld, H. Zhong, R. T. Ruck, M. Shevlin, P. J. Chirik Science 2018, 360, 888-893. (R,R)-iPr-Duphos stands for (R,R)-1,2-bis(2,5-diisopropylphospholan-1-yl)benzene.

### Example 1

### Hydrogenation of (rac)-4,8-dimethyl-1,2,7-nonatrien-4-ol using Co(I) Complex of Formula (I)

A 3 ml glass reactor was charged under argon with (R,R-iPr-Duphos)Co(C₆H₆)][Al(OC(CF₃)₃)₄] (7.8 mg, 0.005 mmol, 0.5 mol %) followed by a solution of (rac)-4,8-dimethyl-1,2,7-nonatrien-4-ol (166.5 mg, 1.002 mmol) dissolved in trifluoromethyl benzene (1 ml). More trifluoromethyl benzene (1 ml) are added to rinse. Then the glass flask is placed in a stainless steel autoclave, which is pressurized with hydrogen (3×10 bar) and then the solution is mechanically stirred under hydrogen (5 bar) at 25° C. for six hours. The autoclave is then vented and the reaction mixture analysed by GC (DB-Wax). Analysis showed a conversion >99% and the formation of (rac)-(Z)-4,8-dimethyl-2,7-nonadien-4-ol in 93%.

### Example 2

### Hydrogenation of (rac)-4,8-dimethyl-1,2,7-nonatrien-4-ol using Co(I) complex of formula (I) in Various Solvent

A 3 ml glass reactor was charged under argon with (R,R)-iPr-Duphos)Co(C₆H₆)][Al(OC(CF₃)₃)₄] (7.8 mg, 0.005 mmol, 0.5 mol %) followed by a solution of (rac)-4,8-dimethyl-1,2,7-nonatrien-4-ol (166.5 mg, 1.002 mmol) dissolved in the desired solvent (2 ml) as shown in Table 1. Then the glass flask was placed in a stainless steel autoclave, which was pressurized with hydrogen (3×10 bar) and then the solution is mechanically stirred under hydrogen (5 bar) at 25° C. for 6 hours. The autoclave was then vented, purged with nitrogen and the reaction mixture analysed by GC (DB-Wax). The results are shown in Table 1.

TABLE 1
Hydrogenation of (rac)-4,8-dimethyl-1,2,7-nonatrien-4-ol
using Co(I) complex of formula (I) in various solvent.¹⁾

| Solvent | Name | Conv.²⁾ | (Z)-product³⁾ |
|---|---|---|---|
| C₄H₈O | tetrahydrofurane | >99 | 68 |
| 2-Me-THF | 2-methyl-tetrahydrofurane | >99 | 69 |
| CF₃C₆H₅ | trifluoromethylbenzene | >99 | 93 |
| 1,2-F₂C₆H₄ | 1,2-difluorobenzene | >99 | 91 |
| 1,2-Cl₂C₆H₄ | 1,2-dichlorobenzene | 93 | 62 |

¹⁾Average of two runs.
²⁾Conversion: 100%-% amount of starting material as measured by GC.
³⁾Amount of (rac)-(Z)-4,8-dimethyl-2,7-nonadien-4-ol as measured by GC.

### Example 3

### Hydrogenation of (rac)-4,8-dimethyl-1,2,7-nonatrien-4-ol using Co(I) Complex of Formula (II)

### General Procedure:

A 3 ml glass reactor was charged under argon successively with [(L)CoCl]₂ (0.0025 mmol, 0.25 mol %), a sodium salt Na⁺X⁻ (0.05 mmol, 5 mol %) followed by a solution of (rac)-4,8-dimethyl-1,2,7-nonatrien-4-ol (2 ml at 0.5M in THF, 1 mmol). Then the glass flask was placed in a stainless steel autoclave, which was purged with hydrogen (3×10 bar) and then the solution was mechanically stirred under hydrogen (5 bar) at 25° C. for 6 hours. The autoclave is then vented, purged with nitrogen and the reaction mixture analysed by GC (DB-Wax). The results obtained with the diphosphines L described in Table 2 are shown in Table 3.

TABLE 2
Diphosphines L used in Table 3.

| Di- | | |
|---|---|---|
| phosphine | Structure | Name |
| L1 | 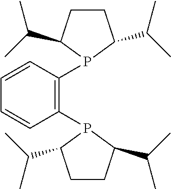 [SMILES: CC(C)[C@H]1CC[C@H](C(C)C)P1c1ccccc1P1[C@@H](C(C)C)CC[C@@H]1C(C)C] | (R,R)-1,2-bis (2,5-diisopropylphospholan- 1-yl)benzene |
| L2 | 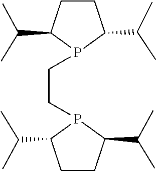 [SMILES: CC(C)[C@H]1CC[C@H](C(C)C)P1CCP1[C@@H](C(C)C)CC[C@@H]1C(C)C] | (R,R)-1,2-bis (2,5-diisopropylphospholan- 1-yl)ethane |
| L3 | 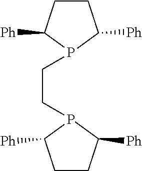 [SMILES: c1ccc([C@H]2CC[C@H](c3ccccc3)P2CCP2[C@@H](c3ccccc3)CC[C@@H]2c2ccccc2)cc1] | (R,R)-1,2-bis (2,5-diphenylphospholan- 1-yl)ethane |

TABLE 3
Hydrogenation of (rac)-4,8-dimethyl-1,2,7-nonatrien-4-ol
using Co(I) complex of formula (II)

| [(L)CoCl]₂ | Na⁺X⁻ | Conv.¹⁾ | (Z)-product²⁾ |
|---|---|---|---|
| L1 | NaBARF³⁾ | >99 | 84 |
| L2 | NaBARF³⁾ | >99 | 82 |
| L3 | NaBARF³⁾ | >99 | 76 |
| L2 | NaSbF₆⁴⁾ | 76 | 65 |

¹⁾Conversion: 100%-amount of starting material as measured by GC.
²⁾Amount of (rac)-(Z)-4,8-dimethyl-2,7-nonadien-4-ol as measured by GC.
³⁾Sodium tetrakis(3,5-bis(trifluoromethyl)phenyl)borate.
⁴⁾Sodium hexafluoroantimonate.
⁵⁾Sodium tetrafluoroborate.

### Example 4

### Hydrogenation of (rac)-4,8-dimethyl-1,2,7-nonatrien-4-ol Using Co(II) Complex of Formula (III) Using LiEt₃BH as Reducing Agent

Under argon, LiEt₃BH (0.03 ml of 1.7 M solution in THF, 0.05 mmol, 0.5 mol %) was added to a solution of (R,R)-1,2-bis(2,5-dimethylphospholan-1-yl)benzene cobalt dichloride (9.9 mg, 0.023 mmol, 0.2 mol %) in THF (2 ml). The purple solution was stirred 5 min and then added to a solution of (rac)-4,8-dimethyl-1,2,7-nonatrien-4-ol (1.67 g, 10 mmol) in THF (20 ml) placed in a stainless steel autoclave. More THF (2×2 ml) was added to rinse. The autoclave was closed, purged with hydrogen (3×5 bar), and the solution was mechanically stirred under hydrogen (5 bar) at 25° C. for 16 hours. The autoclave was then vented, purged with nitrogen and the reaction mixture analysed by GC (DB-Wax). Analysis showed a conversion 99% and the formation of (rac)-(Z)-4,8-dimethyl-2,7-nonadien-4-ol in 66%.

### Example 5

### Hydrogenation of (rac)-4,8-dimethyl-1,2,7-nonatrien-4-ol using Co(II) Complex of Formula (III) Using Zinc as Reducing Agent

Under argon, a stainless steel autoclave was charged successively with (R,R)-1,2-bis(2,5-dimethylphospholan-1-yl)benzene cobalt dichloride (43.8 mg, 0.1 mmol, 1 mol %), zinc powder (132.5 mg, 2.03 mmol, 20 mol %) and methanol (24 ml). The autoclave was closed, purged with hydrogen (3×5 bar), and the solution was mechanically stirred under hydrogen (25 bar) at 50° C. for 21.5 hours. The autoclave was then cooled to room temperature, vented, purged with nitrogen and the reaction mixture analysed by GC (DB-Wax). Analysis showed a conversion >99% and the formation of (rac)-(Z)-4,8-dimethyl-2,7-nonadien-4-ol in 65%.

### Example 6

### Hydrogenation of (rac)-4,8-dimethyl-1,2,7-nonatrien-4-ol using Co(II) Complex of Formula (III) Using NaBH₄ as Reducing Agent

Under argon, NaBH₄ (35.8 mg, 0.95 mmol, 9.5 mol %) was added to a blue suspension of 1,4-bis(diphenylphosphanyl)butane cobalt dichloride (54.6 mg, 0.098 mmol, 1 mol %) in THF (5 ml). The suspension was stirred for 1 h and turned dark brown. Then the solution was added to a solution of (rac)-4,8-dimethyl-1,2,7-nonatrien-4-ol (1.665 g, 10 mmol) in THE (20 ml) placed in a stainless steel autoclave. The autoclave was closed, purged with hydrogen (3×5 bar), and the solution was mechanically stirred under hydrogen (5 bar) at 50° C. for 5 hours. The autoclave was then cooled to room temperature, vented, purged with nitrogen and the reaction mixture analysed by GC (DB-Wax). Analysis showed a conversion >99% and the formation of (rac)-(Z)-4,8-dimethyl-2,7-nonadien-4-ol in 59%.

### Example 7

### Hydrogenation of (rac)-4,8-dimethyl-1,2,7-nonatrien-4-ol Using Co(II) Complex of Formula (III) Using NaBH₄ as Reducing Agent

Under argon, NaBH₄ (35.6 mg, 0.94 mmol, 9.5 mol %) was added to a suspension of 1,4-bis(diphenylphosphanyl)butane cobalt bis pivalate (65.5 mg, 0.095 mmol, 1 mol %) in THF (5 ml). The suspension was stirred for 1 h and turned dark brown. Then the solution was added to a solution of (rac)-4,8-dimethyl-1,2,7-nonatrien-4-ol (1.668 g, 10 mmol) in THF (20 ml) placed in a stainless steel autoclave. The autoclave was closed, purged with hydrogen (3×5 bar), and the solution was mechanically stirred under hydrogen (5 bar) at 50° C. for 4.5 hours. The autoclave was then cooled to room temperature, vented, purged with nitrogen and the reaction mixture analysed by GC (DB-Wax). Analysis showed a conversion >99% and the formation of (rac)-(Z)-4,8-dimethyl-2,7-nonadien-4-ol in 61%.

1. A process for the semi-hydrogenation, using molecular H₂, of an allene into the corresponding alkene wherein said process is carried out in the presence of a a homogeneous catalytic system wherein the homogeneous catalytic system comprises a cobalt salt, a bidentate diphosphine ligand and a weakly or non-coordinating mono anion or a reducing agent.

2. The process according to claim 1, wherein homogeneous catalytic system is a cobalt complex of formula 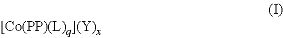
wherein PP is a bidentate diphosphine ligand; x is 0 or 1; L is a C₄-C₂₀ hydrocarbon ligand comprising two or three carbon-carbon double bonds optionally comprising one or more of an oxygen and/or halogen atom and q is 1; or L is C₃-C₁₅ alkene ligand and q is 3; and Y is a weakly or non-coordinating mono anion.

3. The process according to claim 1, wherein homogeneous catalytic system comprises
a) at least a salt of formula MY wherein M is a alkali metal or a alkaline earth metal and Y is a weakly or non-coordinating mono anion; and
b) a Co(I) complex of formula 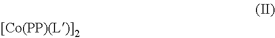
wherein PP is a bidentate diphosphine ligand and L′ is a halogen atom, a C₁₋₁₈ alkoxide, a C₁₋₈ carboxylate or a β-diketonate group.

4. The process according to claim 1, wherein homogeneous catalytic system comprises
a) a reducing agent selected from the group consisting of Zn metal, LiBHEt₃, NaBH₄ and NaBHEt₃; and
b) a Co(II) complex of formula 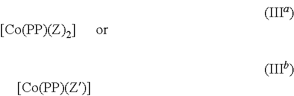
wherein PP is a bidentate diphosphine ligand; Z is a halogen atom, a C₁₋₄ alkyl group optionally substituted by a trimethylsilyl group, a β-diketonate group, a sulfonate group, a phosphate group, a C₁₋₁₈ alkoxide group, a C₁₋₈ carboxylate group or a phenolate group optionally substituted by one or three C₁₋₈ alkyl groups, C₃₋₈ cycloalkyl groups, halogen atoms, C₁₋₃ alkoxy groups, halo- or perhalo-hydrocarbon groups or C₁₋₃ carboxylic ester groups, and Z′ is a carbonate or a sulphate group.

5. The process according to any one of claims 1 to 4, wherein the bidentate diphosphine ligand is a electron rich ligand.

6. The process according to any one of claims 1 to 5, wherein the bidentate diphosphine ligand is of formula 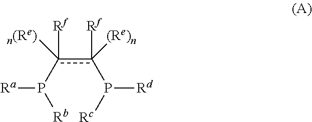
wherein the dotted line is a single bond then n is 1 or the dotted line is a double bond and then n is 0; R^{a}, R^{b}, R^{c} and R^{d}, when taken separately, represent, simultaneously or independently, a linear C₁ to C₈ alkyl, a linear C₂ to C₈ alkenyl, a branched or cyclic C₃ to C₈ alkyl or alkenyl, a C₆ to C₁₀ aryl group, each optionally substituted by one or more of a halogen atom, C₁₋₁₀ alkoxy, hydroxy, halo- or perhalo-hydrocarbon, C₁₋₄ carboxylic ester group; or R^{a} and R^{b}, when taken together, and/or R^{c} and R^{d}, when taken together, form a saturated or unsaturated ring having 4 to 20 atoms and including the phosphorus atom to which said R^{a} and R^{b} groups or R^{c} and R^{d} groups are bonded, said ring being optionally substituted by one or more of a halogen atom, C₁₋₁₀ alkoxy, hydroxy, halo- or perhalo-hydrocarbon, C₁₋₄ carboxylic ester, C₆₋₁₀ aryl group; R^{e} and R^{f}, independently from each other, are a hydrogen atom or a C₁₋₈ hydrocarbon group; optionally comprising one or two oxygen or nitrogen atoms; or both R^{f} are taken together and form a C₃₋₁₀ cycloalkyl, a C₅₋₁₀ cycloalkenyl, a C₄₋₁₀ heterocycloalkyl, a C₄₋₁₀ heterocycloalkenyl, a C₄₋₁₀ heteroaryl or a C₆₋aryl group, each optionally substituted by one or two halogen atoms, C₁₋₁₀ alkoxy, hydroxy, halo- or perhalo-hydrocarbon, C₁₋₄ carboxylic ester groups; or R^{a} and R^{b} and/or R^{d} and R^{f}, when taken together, form a saturated or unsaturated ring, having 4 to 20 atoms and including the phosphorus atom to which said R^{a} and R^{f} groups and/or R^{d} and R^{f} are bonded, said ring being optionally substituted by one or two halogen atoms, C₁₋₁₀ alkoxy, hydroxy, halo- or perhalo-hydrocarbon, C₁₋₄ carboxylic ester groups.

7. The process according to any one of claims 1 to 6, wherein the bidentate diphosphine ligand is of formula 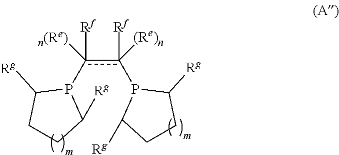
wherein the dotted line, n, R^{e} and R^{f} has the same meaning as defined in claim 6; m is 0, 1 or 2 and each R^{g}, independently from each other, are a C₁₋₈ alkyl or a C₆₋₁₀ aryl group; preferably, R^{g}, independently from each other, are a C₃₋₈ branched alkyl or a phenyl group.

8. The process according to any one of claims 1 to 7, wherein the bidentate diphosphine ligand is selected from the group of 1,2-bis(2,5-diisopropylphospholan-1-yl)benzene, 1,2-bis(2,5-diisopropylphospholan-1-yl)ethane, 1,2-bis(2,5-diphenylphospholan-1-yl)ethane, 1,2-bis(2,5-diphenylphospholan-1-yl)benzene, 1,2-bis(dimethylphosphaneyl)ethane, 1,2-bis(diethylphosphaneyl)ethane, 1,2-bis(diisopropylphosphaneyl)ethane, 1,2-bis(dicyclopentylphosphaneyl)ethane and 1,2-bis(dicyclohexylphosphaneyl)ethane.

9. The process according to any one of claims 1 to 8, wherein the weakly or non-coordinating coordinating mono anion is sterically hindered; preferably, the weakly or non-coordinating coordinating mono anion is Sb⁻F₆ or of formula B⁻R′₄, wherein R′ is a halogen atom or a phenyl group optionally substituted by one to five halogen atoms or methyl or CF₃ groups or of formula Al⁻(OR″)₄ wherein R″ is a C₁₋₆ alkyl optionally substituted by one or more halogen atoms; even more preferably, the weakly or non-coordinating coordinating mono anion is selected from the group consisting of ⁻SbF₆, ⁻BF₄, ⁻Al(OC₃HF₆)₄, ⁻Al(OC₄F₉)₄, ⁻B(3,5-bis(trifluoromethyl)phenyl)₄.

10. The process according to any one of claims 1 to 9, wherein L is an η⁶ ligand selected from the group consisting of (η⁶-C₆H₆), (η⁶-C₆H₅CH₃) (η⁶-C₆H₅OCH₃), (η⁶-C₆H₄(CH₃)₂), (η⁶-C₆H₅CF₃), (η⁶-C₆H₅F), (η⁶-C₆H₄F₂) and (η⁶-C₆H₄(CF₃)₂).

11. The process according to any one of claims 1 to 10, wherein L′ is a chloride atom.

12. The process according to any one of claims 1 to 11, wherein the process is the 1,2-semi-hydrogenation of a terminal allene into the corresponding ω-1 alkene.

13. The process according to any one of claims 1 to 12, wherein terminal allene is of formula 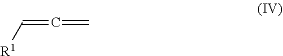
Wherein R¹ is a C₁₋₃₀ hydrocarbon optionally comprising one to three oxygen atoms and/or one to two nitrogen atoms and/or one sulphur atom.

14. The process according to any one of claims 1 to 13, wherein process of the invention is perform in absence of base or additive.

15. Use in the semi-hydrogenation process as defined in claims 1 to 14 of compound being selected from the group consisting of 4,8-dimethylnona-1,2,7-trien-4-ol, 1-(p-tolyl)buta-2,3-dien-1-ol, 1-cyclohexylbuta-2,3-dien-1-ol, undeca-1,2-dien-4-ol, (E)-5-methylocta-1,2,5-trien-4-ol, 1-phenylhexa-4,5-dien-3-ol, (6R)-6,10-dimethylundeca-1,2,9-trien-4-ol, 2-phenylpenta-3,4-dien-2-ol, 3-phenylhexa-4,5-dien-3-ol, 2-methyl-3-phenylhexa-4,5-dien-3-ol, 4-methyldeca-1,2-dien-4-ol, 3-methyl-1-phenylhexa-4,5-dien-3-ol, 4,4-dimethyl-1-(propa-1,2-dien-1-yl)cyclohexan-1-ol and 1-(propa-1,2-dien-1-yl)cyclopentan-1-ol, 1-(propa-1,2-dien-1-yl)cyclohexan-1-ol, 1-(propa-1,2-dien-1-yl)cyclooctan-1-ol and 2-(propa-1,2-dien-1-yl)decahydronaphthalen-2-ol.
